(19) United States
(12) Patent Application Publication
(10) Pub. No.: US 2017/0121317 A1
(43) Pub. Date: May 4, 2017

(54) METHOD FOR PRODUCING COMPOSITIONS OF FURAN GLYCIDYL ETHERS, COMPOSITIONS PRODUCED AND USES OF SAME

(71) Applicant: ROQUETTE FRERES, Lestrem (FR)

(72) Inventors: Clothilde BUFFE, Lomme (FR); Mathias IBERT, La Chapelle D'armentieres (FR)

(21) Appl. No.: 15/311,968

(22) PCT Filed: May 20, 2015
(86) PCT No.: PCT/FR2015/051322

§ 371 Date: Nov. 17, 2016

(30) Foreign Application Priority Data
May 28, 2014 (FR) 1454847

(51) Int. Cl. C07D 407/14; C08G 59/50; C08G 59/26

(52) U.S. Cl. CPC C07D 407/14; C08G 59/26; C08G 59/5026

(57) ABSTRACT

A method for producing a composition of glycidyl ethers synthesised from furan derivatives (furan glycidyl ethers), partly characterized by azeotropic distillation performed under reduced pressure and without the addition of a catalyst. Such products are used to produce epoxy resins, with the aim of forming a three-dimensional macromolecular network. With the compositions of the invention the cross-linking density of the network is increased, allowing the production of a material which is more resistant, both chemically and mechanically, and has a higher glass transition temperature (Tg) than the same materials produced with compositions of furan glycidyl ethers synthesized at atmospheric pressure according to prior art.

The main subject of the present invention is a process for producing a composition of glycidyl ethers synthesized from furan derivatives (in the remainder of the patent application, the term “furan glycidyl ethers” will more simply be used), one of the novel features of which lies in an azeotropic distillation performed under reduced pressure and without the addition of catalyst. Such products are used to produce epoxy resins, with the aim of forming a three-dimensional macromolecular network.

With the compositions according to the invention, the crosslinking density of the network is increased, thereby making it possible to obtain a material that is both more chemically resistant and mechanically stronger and has a higher glass transition temperature (Tg), compared with the same materials obtained with the furan glycidyl ether compositions synthesized at atmospheric pressure according to the prior art.

In addition, the furan glycidyl ether compositions according to the invention result in resins which exhibit a much better water resistance than their homologs produced with the compositions of furan derivatives synthesized at atmospheric pressure according to the prior art.

A subject of the present invention is also the furan glycidyl ether compositions thus produced, and also the uses thereof in the production of composite materials, coatings or else adhesives.

Bisphenol A glycidyl ether (BADGE), of formula (I), is a chemical compound used as crosslinking agent in the production of epoxy resins. This product today appears on the list of IARC (International Agency for Research on Cancer) group 3 carcinogens, that is to say that it is a substance that is not classifiable as to its carcinogenicity to humans.

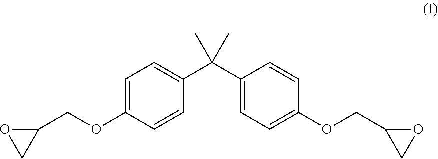 [SMILES: CC(C)(c1ccc(OCC2CO2)cc1)c1ccc(OCC2CO2)cc1]

However, BADGE is in particular used as an additive in coatings for some cans for food. Free BADGE can therefore be found in the content of these cans, thereby raising many questions regarding its carcinogenicity (“Determination of Bisphenol A diglycidyl ether and its hydrolysis products in canned oily foods from the Austrian market”, Z. Lebensm. Unters. Forsch. A 208 (1999) pp. 208-211).

The prior art already provides a certain number of molecules, derived from “green chemistry” (not a slave to petroleum-based raw materials), the structures of which (formulae II, II′ and II″ below) mimic very closely that of BADGE.

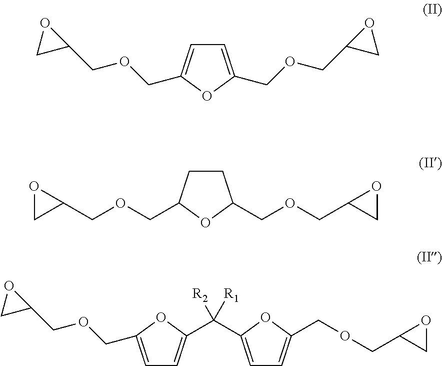

with R₁ and R₂ possibly being a hydrogen and/or an alkyl group.

These compounds, which belong to the more general class of furan glycidyl ethers, are today widely known and described in the literature, as are the processes for the synthesis thereof, which in particular involve respectively the following furan diols: DHMF (2,5-di(hydroxymethyl)furan) and DHMTHF (2,5-di(hydroxymethyl)tetrahydrofuran), the structures of which are given hereinafter (formulae III and III′).

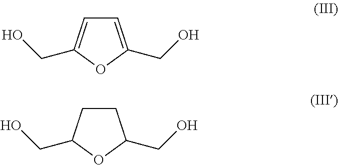

Document US 2012 220742 describes a single-step synthesis process in which a furan derivative is brought into contact with epichlorohydrin. The reaction is carried out at atmospheric pressure, at a temperature in the region of 50° C., with the use of a phase transfer catalyst and in the presence of a THF/water two-phase solvent system. The epoxy derivative is then recovered by water/ethyl acetate liquid-liquid extraction and then by column purification.

Document U.S. Pat. No. 3,025,307 describes a 2-step synthesis process at atmospheric pressure: first of all the furan derivative is brought into contact with epichlorohydrin in the presence of a Lewis acid, and then sodium hydroxide is introduced in the presence of solvent (typically THF).

Document WO 2011 030991 discloses, for its part, a method of synthesis in a solvent medium (THF) by bringing sodium hydroxide, epichlorohydrin and the furan derivative into contact at approximately 50° C. and then by column purification.

More recently, the paper “Synthesis and Characterization of Thermosetting Furan-Based Epoxy Systems” (Macromolecules, Article ASAP, DOI: 10.1021/ma500687t, Publication Date (Web): May 9, 2014) describes a 2-step synthesis at atmospheric pressure. The first step is carried out in the presence of a phase transfer catalyst, then an aqueous sodium hydroxide solution is introduced. The product is recovered by water/diethyl ether liquid-liquid extraction and then purified on a column.

These processes in fact aim to obtain a composition (as opposed to a pure product) containing in particular monofunctional and difunctional derivatives. However, only the difunctional derivatives participate in the formation of the three-dimensional macromolecular network during the production of the resin, in particular in the presence of amine-type curing agents. It is therefore these difunctional derivatives that it will be sought to favor to the detriment of the monofunctional products.

In the same way, care will be taken to limit the content of oligomer (IV) and (IV′) in order to obtain a three-dimensional network having a higher crosslinking density. Indeed, the greater the value of n, the greater the distance between 2 reactive functions and therefore the greater will be the distance between each crosslinking node. A high node density makes it possible to obtain a material which is both more chemically resistant and mechanically stronger and which has a higher glass transition temperature (Tg). The latter property is fundamental for applications where the material synthesized must be used as packaging for “hot” products, such as in particular liquids. This is because, above the glass transition temperature, the material changes to the rubbery state, and its mechanical properties are then changed: it becomes more flexible and deformable.

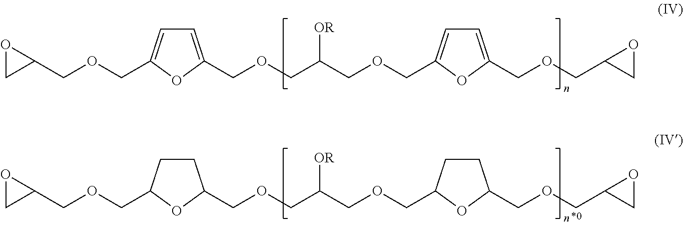

The presence of oligomers and/or of monofunctional derivatives can be directly linked to the epoxy equivalent by weight, defined as the weight of resin containing one glycidyl function equivalent. For example, the diglycidyl ether obtained from DHMF (figure II), which has a molecular weight of 240 g/mol and which contains 2 glycidyl functions, has an epoxy equivalent of 120 g/eq. The higher the epoxy equivalent by weight, the higher the oligomer and/or monofunctional derivative content: it will thus be sought to minimize this epoxy equivalent.

None of the abovementioned prior art documents which target processes for preparing furan glycidyl ethers deals precisely with this complex problem which consists in increasing the selectivity with respect to difunctional derivatives to the detriment of monofunctional derivatives and oligomers. In addition, the abovementioned prior art documents all use organic solvents other than water, while it is today sought to minimize or even prohibit the use of said solvents.

Continuing its research through a very large number of studies, the applicant company has managed to develop a process for producing furan glycidyl ether compositions which in addition has the following advantages:
said process uses only water in terms of organic solvent;
it does not use catalysts, which are often products that are dangerous and/or difficult to eliminate and/or to recycle (for example tetraalkylammonium halides, sulfates or hydrogen sulfates);
the epoxy equivalent of the compositions produced advantageously proves to be lower than that of the prior art compositions;
the glass transition temperature of a resin produced with the compositions of the invention is advantageously higher than that measured for a resin produced with the prior art compositions.

One of the novel features of the process of the invention is that the reaction between the furan derivative (DHMF or DHMTHF) and the organic halide, more specifically the epihalohydrin, is performed under reduced pressure, so as to carry out an azeotropic distillation. Thus, a first subject of the invention consists of a process for producing a composition of furan glycidyl ethers of formula (II) or (II′):

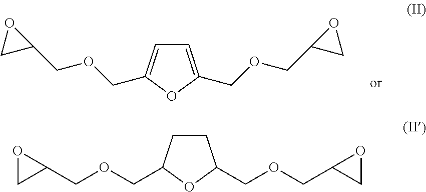

comprising the following steps:
a) bringing the DHMF or the DHMTHF into contact with an organic halide chosen from epibromohydrin, epifluorohydrin, epiiodohydrin and epichlorohydrin,
b) placing the mixture thus obtained under vacuum so as to obtain a low pressure of between 200 and 400 mbar,
c) heating the mixture under vacuum at a temperature of between 50° C. and 120° C. and thus carrying out an azeotropic distillation,
d) then adding to said mixture a basic reagent over a period of between 1 hour and 10 hours and then continuing the azeotropic distillation,
e) recovering the composition after a filtration step, concentration of the filtrate and optionally a purification step.

The first step of the process according to the invention (step a) thus consists in bringing the DHMF or the DHMTHF into contact with an organic halide, more specifically an epihalohydrin.

The organic halide is chosen from epibromohydrin, epifluorohydrin, epiiodohydrin and epichlorohydrin, and is more preferentially epichlorohydrin.

This organic halide is preferentially introduced in excess relative to the hydroxyl functions of the DHMF and of the DHMTHF. Thus, for 1 mol of DHMF or of DHMTHF, preferentially between 2 and 20 mol of organic halide, and more preferentially approximately 10 mol, will be introduced.

This first step is carried out in any device well known to those skilled in the art which makes it possible to bring 2 chemical reagents into contact and which has heating and stirring members. It may for example be a jacketed reactor. The device in question must also be equipped with a member that makes it possible to produce a partial vacuum and with a member that makes it possible to perform an azeotropic distillation, such as an inverse Dean-Stark apparatus surmounted by a reflux condenser.

After this first step of bringing into contact (step a), a partial vacuum is then produced in the device by means of a vacuum pump, the corresponding low pressure being between 100 mbar and 1000 mbar (step b). Said low pressure is preferentially between 200 and 400 mbar and more preferentially between 240 and 280 mbar.

During the third step of the process of the invention (step c), the mixture between the DHMF or the DHMTHF and the organic halide is heated at a temperature of between 50° C. and 120° C. This template is preferentially between 70 and 90° C. and more preferentially between 75 and 85° C.

The temperature of the heat-exchange fluid circulating in the jacket of the reactor must be regulated so as to be at least equal to the boiling point of the organic halide used, so as to begin the azeotropic distillation. During this first distillation phase, said distillation involves only the organic halide: in other words, only a part of the organic halide is eliminated by distillation. Moreover, the boiling point that should be taken into account is the boiling point of the organic halide under the partial pressure which prevails in the device.

By way of example, epichlorohydrin has a boiling point of 116° C. at atmospheric pressure, this boiling point being approximately equal to 80° C. under a partial vacuum of 275 mbar. In practice, the temperature will be a temperature slightly above (approximately 3° C. more) the boiling point for the organic halide under consideration and for the low pressure applied.

During the fourth step of the process of the invention (step d), a basic reagent is then added to the DHMF or DHMTHF/organic halide mixture, for a duration of between 1 hour and 10 hours, preferentially between 1 h and 6 h and more preferentially between 2 h and 4 h.

The amount of basic reagent is preferentially the stoichiometric amount relative to the number of hydroxyl functions of the DHMF or DHMTHF (for example: 2 mol of sodium hydroxide per 1 mol of furan derivative). It is nevertheless possible to choose to use a slight excess relative to this stoichiometry.

The basic reagent is chosen from lithium hydroxide, potassium hydroxide, calcium hydroxide and sodium hydroxide, optionally in the form of an aqueous solution, and is very preferentially an aqueous sodium hydroxide solution.

As soon as the basic reagent is introduced (step d), there is formation of water by reaction between the DHMF or DHMTHF and the organic halide, and likewise additional water may be contributed thereto by introducing the basic reagent in the form of an aqueous solution. The distillation then involves the mixture of water and organic halide, the first being eliminated and the second returning to the reaction medium. In the case of a Dean-Stark apparatus: the water constitutes the upper phase which is eliminated, whereas the halide in the lower part is returned to the reaction medium.

The azeotropic distillation is continued until the water is completely eliminated. Thus, the reaction medium is again heated for a period of between 30 minutes and 1 hour after the end of the addition of the basic reagent.

The reaction medium is finally filtered in order to eliminate the salts formed during the reaction between the halide and the DHMF or DHMTHF, such as sodium chloride in the case of epichlorohydrin. The salts thus recovered are washed once again with epichlorohydrin. The washing liquors are added to the first filtrate and then concentrated so as to eliminate in particular the epichlorohydrin. The concentration step is carried out for example by vacuum distillation, for example in a device of rotary evaporator and/or scraped film evaporator type. During this concentration step, the crude product or the composition is gradually heated to 140° C. and the pressure is reduced to 1 mbar.

Optionally, an additional step of purification by distillation under reduced pressure (<1 mbar) can be carried out by means of a scraped surface exchanger in order to separate the oligomers from the monofunctional derivatives. This step is distinct from that described in the preceding paragraph.

Another subject of the present invention is based on the compositions that can be obtained according to the process of the invention.

A final subject of the present invention is based on the use of these compositions for the production of composite materials, coatings and adhesives.

These compositions can also be used for the synthesis of vinyl ester by reaction with (meth)acrylic acids. These photo-crosslinkable monomers (vinyl esters) may then be used for the production of dental resins, boat hulls and speciality coatings.

The compositions according to the invention can be used in polycondensation reactions in order to obtain a three-dimensional network and a thermorigid material.

In this case, they can be used alone (homopolymerization reactions) or in combination with other monomers (copolymerization reactions).

Among the comonomers, mention may be made of the other epoxy derivatives, but also agents referred to as curing agents or crosslinking agents, such as amines, polyetheramines, polyamides, amidoamines, Mannich bases, anhydrides, polycarboxylic polyesters, mercaptans, phenolic resins, melamine-, urea- and phenol-formaldehyde resins. Catalysts of Lewis acid, tertiary amine or imidazole type can also be added to the formulation in order to initiate and/or accelerate the crosslinking. The crosslinking reactions will be carried out at a temperature ranging from 5° C. to 260° C.

The materials, resins, obtained from the furan glycidyl ether compositions, which are the subject of the present invention, are more chemically resistant and mechanically stronger and also have a higher glass transition temperature (Tg), compared with the same materials obtained with the furan glycidyl ether compositions according to the prior art, as demonstrated hereinafter.

### EXAMPLES

Reagents:

DHMF: sold by the company Pennakem.

DHMTHF: obtained after hydrogenation of DHMF (95%, Pennakem) over Raney Ni at 110° C. and 70 bar, then purification by distillation.

Epichlorohydrin: sold by the company Sigma-Aldrich.

Tetraethylammonium bromide: sold by the company Sigma-Aldrich.

Tetrabutylammonium bromide: sold by the company Sigma-Aldrich.

### Example 1: Tests According to the Prior Art

3 tests were carried out:
2 (tests 1 and 2) during which, respectively, the DHMF and the DHMTHF and the epichlorohydrin were reacted, with addition of an aqueous sodium hydroxide solution, the azeotropic distillation being carried out at atmospheric pressure;
1 according to the conditions of document WO 2011 030991 mentioned above.

Test No. 1

50 g of DHMF (0.39 mol, 1 molar equivalent) and then 361.2 g of epichlorohydrin (3.90 mol, 10 molar equivalents) are introduced into a 1-liter jacketed reactor equipped with a thermostatic bath containing a heat-exchange fluid, with a mechanical blade stirring system, with a system for controlling the temperature of the reaction medium and with an inverse Dean-Stark apparatus surmounted by a reflux condenser.

The reaction mixture is then heated to 116° C. (boiling point of epichlorohydrin=116° C. at atmospheric pressure) over 30 minutes.

125 g of a 50% aqueous sodium hydroxide solution (1.56 mol, 2 molar equivalents) are then gradually added.

The addition lasts for a total of 1 h 40; the azeotropic distillation continues and the water formed by reaction between the halide (in the case in point the epichlorohydrin) and the DHMF is eliminated.

The reaction medium is filtered under vacuum in order to eliminate therefrom the sodium chloride formed over time. The salts are finally washed with epichlorohydrin which is then eliminated by evaporation under reduced pressure on a rotary evaporator.

The composition of furan diglycidyl ether or containing predominantly furan diglycidyl ether is then obtained in the form of a liquid (Brookfield viscosity at 25° C. of 442 mPa·s), having an epoxy equivalent of 189 g/equivalent.

Test No. 2

The process was carried out in the same way as for test No. 1, with the difference that the starting material used, the furan derivative, is DHMTHF.

The composition of furan diglycidyl ether or containing predominantly furan diglycidyl ether is then obtained in the form of a liquid (Brookfield viscosity at 25° C. of 69 mPa·s), having an epoxy equivalent of 162 g/equivalent.

Test No. 3

This test is carried out under the conditions of document WO 2011 030991, example 1, and therefore uses an organic solvent other than water.

To do this, 252.8 g of epichlorohydrin (14 molar equivalents) and then 187.4 g of a 50% aqueous sodium hydroxide solution (12 molar equivalents) and 4.10 g of tetrabutylammonium bromide are introduced into a 1-liter jacketed reactor equipped with a thermostatic bath containing a heat-exchange fluid, with a mechanical blade stirring system, with a system for controlling the temperature of the reaction medium and with an inverse Dean-Stark apparatus surmounted by a reflux condenser.

A solution containing 25 g of DHMF (1 molar equivalent) and 293 ml of tetrahydrofuran (THF) is gradually added. The reaction medium is kept stirring at 50° C. for 2 h.

A water-ethyl acetate liquid-liquid extraction is carried out. The aqueous phase is extracted twice with ethyl acetate. The organic phases are dried with anhydrous magnesium sulfate and the resulting product is concentrated on a rotary evaporator under reduced pressure.

The composition of furan diglycidyl ether or containing predominantly furan diglycidyl ether is then obtained in the form of a liquid having an epoxy equivalent of 405 g/equivalent.

Table 1 reproduces the operating conditions, and in particular:
the organic solvent(s) used;
the amount of epichlorohydrin used, expressed in molar equivalent of epichlorohydrin relative to the number of moles of furan derivative (Mol Eq EPI);
the amount of sodium hydroxide used, expressed in molar equivalent of sodium hydroxide relative to the number of moles of furan derivative (Mol Eq NaOH);
the sodium hydroxide introduction time (NaOH intro time).

TABLE 1

| Test No. | 1 | 2 | 3 |
|---|---|---|---|
| solvent | water | water | water/THF/ |
| | | | ethyl acetate |
| Mol Eq EPI | 10 | 10 | 14 |
| Mol Eq NaOH | 2 | 2 | 12 |
| NaOH intro time | 3 h 35 | 3 h 32 | |
| Epoxy Equivalent (g/eq) | 189 | 162 | 405 |

### Example 2: Tests According to the Invention

2 tests according to the invention (tests 4 and 5) were carried out during which the DHMF or the DHMTHF and the epichlorohydrin were reacted, with addition of an aqueous sodium hydroxide solution, the azeotropic distillation being carried out under a partial vacuum.

Test No. 4

50 g of DHMF (0.39 mol, 1 molar equivalent) and then 361.2 g of epichlorohydrin (3.9 mol, 10 molar equivalents) are introduced into a 1-liter jacketed reactor equipped with a thermostatic bath containing a heat-exchange fluid, equipped with a mechanical blade stirring system, with a system for controlling the temperature of the reaction medium and with an inverse Dean-Stark apparatus surmounted by a reflux condenser.

The system is brought to a pressure of 275 mbar relative. The reaction mixture is then heated to 80° C. (boiling point=80° C. at 275 mbar) over 30 minutes before beginning the controlled addition of 125 g of a 50% aqueous sodium hydroxide solution (1.56 mol, 2 molar equivalents). The addition lasts a total of 3 h 35. The water is continuously eliminated by azeotropic distillation.

The reaction medium is filtered under vacuum in order to eliminate therefrom the sodium chloride formed over time. The salts are washed with epichlorohydrin which is then eliminated by evaporation under reduced pressure on a rotary evaporator.

The composition of furan diglycidyl ether or containing predominantly furan diglycidyl ether is then obtained in the form of a clear liquid (Brookfield viscosity at 25° C. of 139 mPa·s), having an epoxy equivalent of 137 g/equivalent.

Test No. 5

The process was carried out in the same way as for test No. 4, with the difference that the starting material used is DHMTHF.

The composition of furan diglycidyl ether or containing predominantly furan diglycidyl ether is then obtained in the form of a liquid (Brookfield viscosity at 25° C. of 31 mPa·s), having an epoxy equivalent of 139 g/equivalent.

TABLE 2

| Test No. | 4 | 5 |
|---|---|---|
| Mol Eq EPI | 10 | 10 |
| Mol Eq NaOH | 2 | 2 |
| NaOH intro time | 3 h 35 | 3 h 35 |
| Pressure (mbar) | 275 | 275 |
| Epoxy Equivalent (g/eq) | 137 | 139 |

Table 3 indicates the distribution determined by GC (as % of surface area) of the various constituents of the final product.

In all the examples of the present application, the GC analysis is carried out on a capillary column of DB1 type (30 m×0.32 mm, film thickness of 0.25 μm). The quantification of the species consists in calculating the relative proportion of the areas of the peaks of the chromatogram, the % of each species (x) being the area of the peak of the species (x) divided by the sum of the area of all the peaks.

TABLE 3

Test No.

| | 1 | 4 |
|---|---|---|
| | Outside | According to |
| | the invention | the invention |
| DHMF | 0.9% | 0% |
| DHMF monoglycidyl ether | 26.3% | 3.8% |
| DHMF diglycidyl ether | 27% | 79.6% |
| di-DHMF monoglycidyl ether | 4% | 0% |
| di-DHMF diglycidyl ether | 5.1% | 6.4% |
| others | 36.7% | 10.2% |
| % DHMF diglycidyl ether/ | 51% | 95.4% |
| (DHMF mono + diglycidyl ether) | | |

The comparison between tests 1 and 4 demonstrates that the process according to the invention makes it possible to obtain compositions in which the proportion of furan diglycidyl ether relative to the furan monoglycidyl ether is considerably higher.

### Example 3: Preparation of Resins from Compositions According to the Invention or According to the Prior Art

Epoxy resins starting from the above compositions and in the presence of a curing agent of amine type (isophorone diamine) were prepared.

The amount of isophorone diamine introduced is calculated so that the ratio of the number of —NH groups to the number of epoxy groups is equal to 1.

Isophorone diamine is available under the brand name Vestamid® IPD from Evonik. The —NH group equivalent by weight is 42.5 g/eq. The formula used to calculate the uses of diamine is the following:

$$w ( isophorone diamine ) = \frac{w epoxy \times 42.5}{Epoxy equivalent}$$

By way of example, the procedure used for test 6 is explained below.

5.06 g of the product obtained in test No. 1 are mixed at ambient temperature with 1.1589 g of isophorone diamine for 1 minute. The mixture, which is homogeneous and flows at ambient temperature, is placed in a silicone mould (L=43 mm, W=20 mm). The crosslinking is carried out in an oven for 1 hour at 80° C. and 2 h at 180° C.

A material which is solid at ambient temperature and which has a glass transition temperature (Tg) of 27° C. is then obtained. The glass transition temperature is measured by DSC at the second pass of a temperature gradient from −100 to 200° C. at 10° C./min.

In addition, the percentage by weight of absorbed water was also determined, by double weighing before and after immersion of each composition in water for 24 hours.

Table 4 reproduces the results obtained according to the furan glycidyl ether compositions used.

TABLE 4

| Test No. | 6 | 7 | 8 | 9 | 10 |
|---|---|---|---|---|---|
| Starting | DHMF | DHMTHF | DHMF | DHMF | DHMTHF |
| material | | | | | |
| Reference of | 1 | 2 | 3 | 4 | 5 |
| the test | (outside | (outside | (outside | (according | (according |
| corre- | the | the | the | to the | to the |
| sponding | invention) | invention) | invention) | invention) | invention) |
| to the furan | | | | | |
| glycidyl | | | | | |
| ether used. | | | | | |
| Epoxy | 189 | 162 | 403 | 137 | 139 |
| equivalent | | | | | |
| (g/eq) | | | | | |
| Tg (° C.) | 27 | 20 | 20 | 69 | 45 |
| % absorbed | 6.1 | 22.2 | 43.3 | 0.6 | 16.7 |
| water | | | | | |

For one and the same starting raw material, a clear decrease in the epoxy equivalent and, in parallel, a strong increase in the transition temperature along with a decrease in water uptake are noted, this being for the tests carried out with the compositions according to the invention in comparison with the tests with the compositions of the prior art.

1. A process for producing a composition of furan glycidyl ethers of formula (II) or (II′): 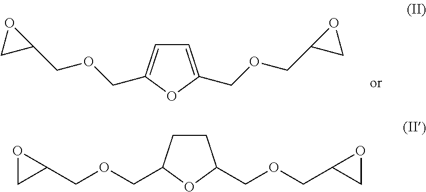
comprising the following steps:
a) bringing the 2,5-di(hydroxyméthyl)furan (DHMF) or the 2,5-di(hydroxymethyl)tetrahydrofuran (DHMTHF) into contact with an organic halide selected from the group consisting of epibromohydrin, epifluorohydrin, epiiodohydrin and epichlorohydrin,
b) placing the mixture thus obtained under vacuum so as to obtain a low pressure of between 200 and 400 mbar,
c) heating the mixture under vacuum at a temperature of between 50° C. and 120° C. and thus carrying out an azeotropic distillation,
d) then adding to said mixture a basic reagent over a period of between 1 hour and 10 hours and then continuing the azeotropic distillation,
e) recovering the composition after a filtration step, concentration of the filtrate and optionally a purification step.

2. The process as claimed in claim 1, wherein the organic halide is epichlorohydrin.

3. The process as claimed in claim 1, wherein the organic halide is introduced in excess relative to the hydroxyl functions of the DHMF and of the DHMTHF.

4. The process as claimed in claim 1, wherein the low pressure during step b) is between 240 and 280 mbar.

5. The process as claimed in claim 1, wherein the temperature during step c) is between 70 and 90° C.

6. The process as claimed in claim 1, wherein the duration during step d) is between 1 h and 6 h.

7. The process as claimed in claim 1, wherein the basic reagent is selected from the group consisting of lithium hydroxide, potassium hydroxide, calcium hydroxide and sodium hydroxide.

8. Compositions that can be obtained according to the process as claimed in claim 1.

9. Method for the production of composite materials, coatings and adhesives, and for the synthesis of vinyl ester which comprises reacting the compositions as claimed in claim 8 with (meth)acrylic acids.

10. The process as claimed in claim 2, wherein the organic halide is introduced in excess relative to the hydroxyl functions of the DHMF and of the DHMTHF.

11. The process as claimed in claim 2, wherein the low pressure during step b) is between 240 and 280 mbar.

12. The process as claimed in claim 5, wherein the temperature during step c) is between 75 and 85° C.

13. The process as claimed in claim 6, wherein the duration during step d) is between 2 h and 4 h.

14. The process as claimed in claim 7, wherein the basic reagent is in the form of an aqueous solution.

15. The process as claimed in claim 14, wherein the aqueous solution is an aqueous sodium hydroxide solution.
